(19) United States
(12) Patent Application Publication
(10) Pub. No.: US 2010/0298821 A1
(43) Pub. Date: Nov. 25, 2010

(54) DEVICE AND METHOD FOR THE THERMAL ABLATION OF TUMORS BY MEANS OF HIGH-FREQUENCY ELECTROMAGNETIC ENERGY UNDER OVERPRESSURE CONDITIONS

(76) Inventor: Giberto Garbagnati, Milano (IT)

Correspondence Address: Steinfl & Bruno, 301 N Lake Ave Ste 810, Pasadena, CA, 91101, US

(21) Appl. No.: 12/295,207

(22) PCT Filed: Mar. 31, 2006
(86) PCT No.: PCT/IT2006/000209

§ 371 Date: Aug. 5, 2009

(51) Int. Cl. A61B 18/18

(52) U.S. Cl. 606/33

(57) ABSTRACT

A device for the TA by means of high frequency comprising a thin hollow element (1) and one or more electrodes (4) being arranged in proximity of the tip (2) of said hollow element (1) and connected to an electromagnetic energy generator set at high frequencies, e.g. radiofrequencies or microwaves, wherein said hollow element (1) is tightly inserted into an expandable balloon (3). Said balloon (3) transmits to the tumoral tissues surrounding it a pressure being higher than the atmospheric one, thus increasing their boiling temperature. The invention also relates to a method for the TA by means of high frequency under overpressure conditions, employing the above-mentioned device.

The present invention relates to a device and a method for the treatment of tumors by means of thermal ablation (TA) induced by electromagnetic energy, e.g. in the radiofrequencies (RF) or in the microwaves (MW) range, and particularly to a device and a method for the TA under overpressure conditions.

It is known that the procedure of TA induced by electromagnetic energy essentially consists of inserting into a tumoral mass an electrode that, being supplied with electromagnetic energy at a suitable frequency, leads to the generation of heat in the tumoral tissues surrounding the electrode, thus causing their coagulative necrosis. The electrode, being generally placed at the end of a needle or a catheter, is percutaneously introduced in the mass of the tumor and it is guided by means of echography or other visualization technique known in the art. This procedure has proved to be effective for the ablation of tumors of the liver and it has recently been suggested also for the ablation of tumors of lung, kidney and other parenchymal organs.

One of the problems inherent in this kind of procedure resides in the difficulty of destroying tumoral masses having a diameter that is larger than about 3 cm. The main reason is that the energy delivered through the electrode inserted in the tumoral mass cannot be indefinitely increased. In fact, if on the one hand the delivery of high power allows to increase the size of the thermal lesion, on the other hand it results in a rapid dehydration of the tissue being closest to the electrode. This causes a rapid increase in the electrical impedance, resulting in the impossibility of delivering further energy to said surrounding tissue. Another problem of the known art is that it is not possible to control the shape of the generated thermal lesion, along with the risk of generating thermal lesions poorly corresponding to the shape of the tumor being treated.

Devices and methods are already known for delaying the dehydration of the tissues being adjacent to the electrode, providing for the use of an expandable balloon. For instance, U.S. Pat. No. 6,952,615 discloses a catheter provided with a balloon arranged at its end and with electrodes placed inside the balloon. Inside the balloon a conductive liquid is contained, which is evenly heated by the electrodes by means of suitable temperature homogenization means. The tissues contacting the balloon undergo the coagulative necrosis. The use of the balloon allows to obtain thermal lesions having an regular and predictable shape, however the volume of the generated thermal lesions is rather limited as heat is transmitted to the tissue liquids by means of conduction only.

In patent application WO 9428809 a device is disclosed being provided with an electrode that is cooled by means of a cooling system based on the circulation of a fluid. The cooled electrode delays the dehydration of the tissues being adjacent thereto which is due to high temperatures, thus allowing to generate thermal lesions having a larger volume with respect to those achievable without cooling. However, even in a longer time, the dehydration of the tissues anyway occurs with such a device, but only thermal lesions having a limited volume can be achieved due to the energy delivery interruption caused by the sudden increase of the impedance.

Object of the present invention is thus to provide a device and a method for the TA being free from the above-mentioned drawbacks, being suitable for increasing the volume of the thermal lesion to the utmost and for giving it a shape that is as round as possible. Such an object is achieved with the device for the TA according to the present invention, whose characteristics are specified in claim 1. Further characteristics of such a device are specified in the dependent claims. In the subsequent claims the characteristics of the method for the TA according to the present invention are specified.

According to the present invention, by means of an increase in the pressure on the tissues it is possible to obtain an increase in the boiling temperature of the tissue liquids, and thereby deliver more energy thereto and heat the zone being affected by the tumor for a longer time, thus reaching regions being farther from the electrode.

An advantage of the device and the method for the TA according to the present invention is that it can be combined with any type of TA electrode, such as, for example, electrodes provided with conductive filaments, cooled electrodes, bipolar electrodes, combinations thereof, and it can be used for the TA by microwaves.

This and other advantages of the device for the TA according to the present invention will be evident to those skilled in the art from the following detailed description of some embodiments thereof with reference to the annexed drawings wherein:

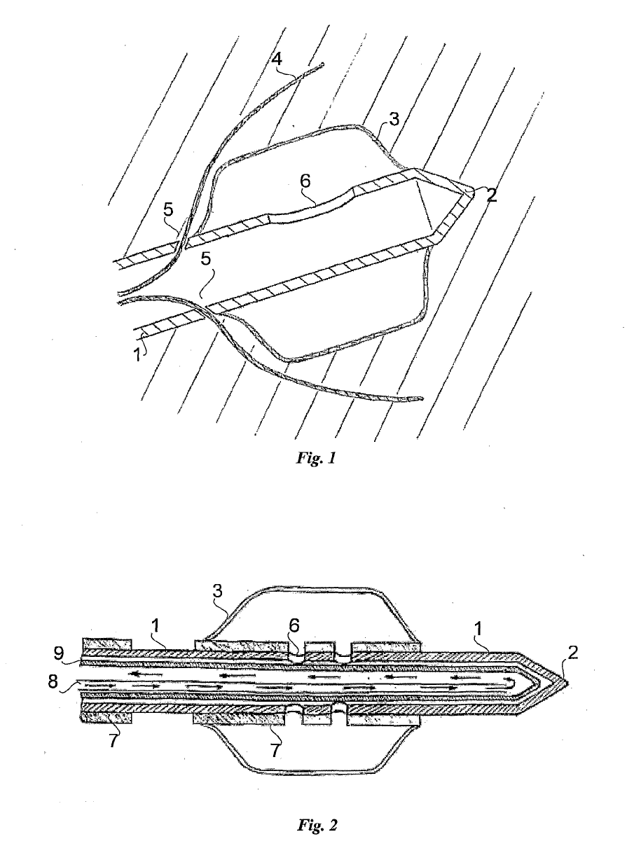

FIG. 1 shows a detailed sectional view of the end of the hollow element according to a first embodiment of the device for the TA being inserted in the mass of the tumor;

FIG. 2 shows a detailed sectional view of the end of the hollow element of a second embodiment of the device for the TA being inserted in the mass of the tumor;

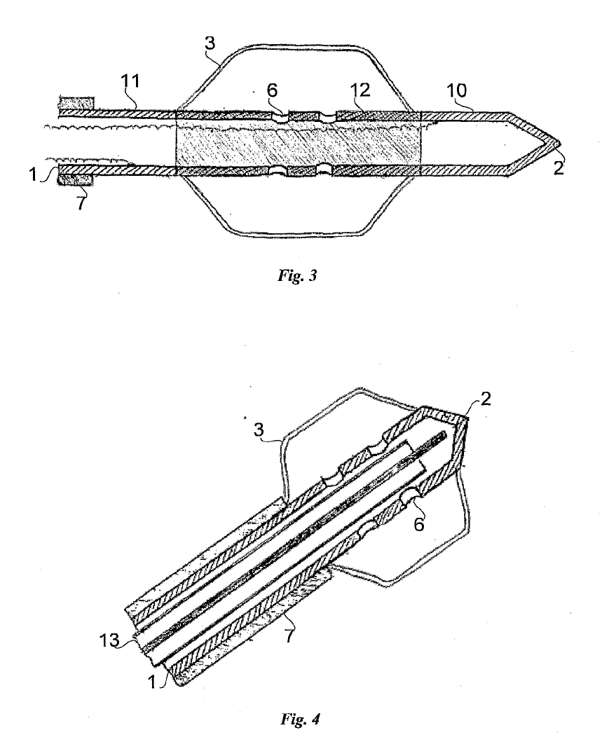

FIG. 3 shows a detailed sectional view of the end of the hollow element according to a third embodiment of the device for the TA; and

FIG. 4 shows a detailed sectional view of the end of the hollow element according to a fourth embodiment of the device for the TA.

The inventor started from the observation that in known TA procedures with RF, by using needles having an increasing diameter, in the proximity of the needle temperatures have been measured being increasing as well and being higher than the boiling temperature of water at atmospheric pressure. On the basis of this observation it has been supposed that the compression caused by the needle presence results in increasing the pressure in the liquids contained in the tissues being adjacent thereto.

The device according to the present invention is provided with a small-caliber spiky element and with an expandable balloon capable of locally pressing the tissue to be treated, thus increasing its pressure.

In FIG. 1 an embodiment of the TA device according to the present invention is shown, being suitable for delivering radiofrequency electromagnetic energy. The device comprises a thin hollow, element 1, as for instance a needle or a catheter, having a closed tip 2, said element being suitable for penetrating the tissues to be subject to a TA procedure, an expandable balloon 3 connected to the hollow element 1, said balloon being made of a biocompatible material resistant to temperatures up to at least 180° C. and suitable for pressing the tissues so as to generate thereon a pressure being higher than the atmospheric one. Preferably, the expandable balloon 3 is coaxially assembled on the hollow element 1 and sealed thereon in proximity of its tip 2. The balloon can have any shape, preferably cylindrical, with suitable zones of connection to the hollow element 1. The device further includes one or more filiform electrodes 4 suitably constrained to the hollow element 1, being connected to a radiofrequency electromagnetic energy generator. The electrodes 4 are movable with respect to the hollow element 1, and are extracted from its main body through one or more corresponding openings 5 that are circumferentially arranged in proximity of the connection of the balloon 3 to the hollow element 1. In order to ease the inserting operation of the hollow element 1 into the tissues to be treated, the electrodes 4 are extracted only after such a step. Still in order to ease the insertion, the balloon 3 is initially deflated. Once the hollow element 1 has been inserted, the electrodes 4 are extracted thus contacting the tissues, then an injection system delivers a fluid into the balloon 3 through one or more openings 6 formed in proximity of the tip 2 of the hollow element 1. The balloon 3 expands thus pressing the tissues being close to the electrodes 4 until a pressure is achieved which is higher than the atmospheric one and which is suitable for obtaining an increase in the boiling temperature of the tissue liquids. The pressure generated by the balloon 3 on the surrounding tissues can be measured with transducers known in the art, and feedback controlled in order to grant the constancy of the parameters throughout the procedure.

Once the hollow element 1 and the electrodes 4 have been arranged and the balloon 3 has been pressurized, the electromagnetic energy generator supplies the electrodes 4 thus causing ionic turbulence in the liquids contained in the tissues and thereby resistive heat. All tissues being comprised between the electrodes and the 60° C. isotherm undergo a non-reversible coagulative necrosis. Non-reversible damages are associated to temperatures comprised between 46° C. and 60° C., whose entity is proportional to the time of exposure.

The compression of the tissues by means of the balloon 3 has the effect of increasing the boiling temperature of the liquids contained in the same tissues, thereby in these conditions the electrodes 4 can supply larger amounts of energy to the tissues. In order to make the increase in the temperature of the tissue liquids take place where the electromagnetic energy is delivered, the balloon can be positioned anywhere as long as close to the electrode. Delivering high power for a longer time allows to obtain the coagulative necrosis in regions which are farther from the electrode and thereby to obtain thermal lesions having a much larger volume. The process stops only when the dehydration of the tissues is complete in the zone being close to the hollow element 1, and thereby it is impossible to deliver further energy to the tissues. This happens, depending on the pressure exerted by the device, at temperatures being higher than 100° C., which until now were unreachable with the TA devices known in the art.

The presence of one or more filiform electrodes 4 allows to distribute the delivered energy in an even way in more directions, with the aim of generating spherically shaped thermal lesions that resemble the shape and size of the mass of the tumor being treated. In other embodiments (not shown) the filiform electrodes 4 can be directly arranged on the external surface of the balloon 3, thus avoiding possible complications when extracting them from the body of the hollow element 1.

In FIG. 2 an alternative embodiment of the device for the TA with RF according to the present invention is shown, wherein the hollow element 1 is made of a conductive material and is connected to an electromagnetic energy generator thus being the electrode. The hollow element 1 is partially covered by an insulating sheath 7. The expandable balloon 3 is fixed on a portion of the isolating sheath 7 in order to avoid the overheating of the same balloon during the delivery of electromagnetic energy. The exposed conductive portions of the hollow element 1 preferably have a total length comprised between about 1 mm and about 100 mm, depending on the type and the size of the thermal lesion desired to be produced. In this embodiment, the device is also cooled by means of a cooling system based, e.g., on the circulation of a cooling fluid 8. The circulation can, for instance, take place inside a cooling circuit 9, e.g. a stylet, which is inserted into the hollow element 1. Alternatively, a catheter injecting the cooling fluid 8 can be inserted with play into the hollow element 1. The fluid can thereby flow off between the catheter external walls and the internal walls of the hollow element 1 thus absorbing heat.

In FIG. 3 another embodiment of the device for the TA with RF according to the present invention is shown, being of a bipolar type. The end of the hollow element 1 is divided into an upper zone 10 and a lower zone 11 by interposing a ring 12 made of insulating material and having diameter and thickness equal to the element 1. The two upper 10 and lower 11 zones are connected to the two poles of the circuit, thus forming the active electrode and the counter electrode respectively. The opening or openings 6 are formed in the ring 12, and the balloon 3 is coaxially assembled on the hollow element 1 and sealed on the ring 12.

In FIG. 4 a further embodiment of the TA device according to the present invention is shown, being of a microwaves type. Similarly to the previous embodiments, the hollow element 1 is provided with a balloon 3 being coaxially assembled on the hollow element 1 and with openings 6 circumferentially arranged in proximity of the tip 2. However, in this case a coaxial cable 13 is inserted into the hollow element 1, delivering electromagnetic energy in the microwaves range. In this case the hollow element 1 is formed of materials being transparent to microwaves, in order not to interfere with their propagation through the tissues.

Suitable materials for manufacturing the semipermeable balloon are, for example, polymeric materials based on PET, PP, PA or PE, or elastomeric materials such as silicon or cured rubber.

Possible variants and/or additions may be made by those skilled in the art to the embodiments described above and illustrated in the annexed drawings while remaining within the scope of the same invention.

By means of the above-described devices it is possible to advantageously perform the method for the TA according to the present invention, comprising the steps of:
a. inserting into a tumoral mass a device provided with a hollow element 1, one or more electrodes 1, 4 and an expandable balloon 3;
b. pressurizing the balloon 3 by means of the injection of a fluid; and
c. delivering high frequency electromagnetic energy to the tumoral mass till the coagulative necrosis of the tissues;
   and wherein said balloon 3 transfers to the tumoral tissues surrounding it a pressure being higher than the atmospheric one.

1.-13. (canceled)

14. A device for thermal ablation, comprising
a hollow element;
one or more electrodes arranged in proximity to a tip of the hollow element and suitable for being connected to a high frequency electromagnetic energy generator;
an expandable balloon connected to said hollow element, the expandable balloon made of a biocompatible material resistant to temperatures higher than 180° C. and suitable to be inflated by a fluid injected therein through one or more openings formed on a portion of said hollow element connected to said balloon, the expandable balloon being suitable for transmitting to tumoral tissues surrounding the expandable balloon a pressure higher than an atmospheric pressure; and
pressure transducers suitable to allow feedback control of the pressure transmitted by the balloon to said tumoral tissues.

15. The device of claim 14, wherein the expandable balloon is made of polymeric materials based on PET, PP, PA and/or PE and/or elastomeric materials, such as silicone materials or cured rubber.

16. The device of claim 14, wherein the expandable balloon is coaxially assembled on the hollow element and sealed on the hollow element in proximity to the tip of the hollow element.

17. The device of claim 14, wherein the hollow element includes a cooling circuit suitable for circulating a cooling fluid.

18. The device of claim 16, wherein the hollow element includes a cooling circuit suitable for circulating a cooling fluid.

19. The device of claim 14, wherein said one or more electrodes are extractable from the hollow element through one or more corresponding openings circumferentially arranged on the hollow element in proximity to the expandable balloon.

20. The device of claim 16, wherein said one or more electrodes are extractable from the hollow element through one or more corresponding openings circumferentially arranged on the hollow element in proximity to the expandable balloon.

21. The device of claim 17, wherein said one or more electrodes are extractable from the hollow element through one or more corresponding openings circumferentially arranged on the hollow element in proximity to the expandable balloon.

22. The device of claim 18, wherein said one or more electrodes are extractable from the hollow element through one or more corresponding openings circumferentially arranged on the hollow element in proximity to the expandable balloon.

23. The device of claim 14, wherein the hollow element is made of a conductive material and is connected to said high frequency electromagnetic energy generator, thus forming an electrode.

24. The device of claim 16, wherein the hollow element is made of a conductive material and is connected to said high frequency electromagnetic energy generator, thus forming an electrode.

25. The device of claim 17, wherein the hollow element is made of a conductive material and is connected to said high frequency electromagnetic energy generator, thus forming an electrode.

26. The device of claim 18, wherein the hollow element is made of a conductive material and is connected to said high frequency electromagnetic energy generator, thus forming an electrode.

27. The device of claim 14, wherein
an end of the hollow element comprises an upper zone and a lower zone separated by a ring made of insulating material and having diameter and thickness equal to the hollow element, said upper and lower zones being connected to the two poles of an electric circuit, and
said balloon is coaxially assembled on the hollow element and sealed on said ring.

28. The device of claim 16, wherein
an end of the hollow element comprises an upper zone and a lower zone separated by a ring made of insulating material and having diameter and thickness equal to the hollow element, said upper and lower zones being connected to two poles of an electric circuit, and
said balloon is coaxially assembled on the hollow element and sealed on said ring.

29. The device of claim 17, wherein
an end of the hollow element comprises an upper zone and a lower zone separated by a ring made of insulating material and having diameter and thickness equal to the hollow element, said upper and lower zones being connected to two poles of an electric circuit, and
said balloon is coaxially assembled on the hollow element and sealed on said ring.

30. The device of claim 18, wherein
an end of the hollow element comprises an upper zone and a lower zone separated by a ring made of insulating material and having diameter and thickness equal to the hollow element, said upper and lower zones being connected to two poles of an electric circuit, and
said balloon is coaxially assembled on the hollow element and sealed on said ring.

31. The device of claim 14, wherein said one or more electrodes comprise a microwave coaxial cable inserted in the hollow element.

32. The device of claim 16, wherein said one or more electrodes comprise a microwave coaxial cable inserted in the hollow element.

33. The device of claim 17, wherein said one or more electrodes comprise a microwave coaxial cable inserted in the hollow element.

34. The device of claim 18, wherein said one or more electrodes comprise a microwave coaxial cable inserted in the hollow element.

35. A method for the thermal ablation including the steps of:
inserting into a tumoral mass a device provided with a hollow element and one or more electrodes, said hollow element being connected to an expandable balloon;
pressurizing the expandable balloon by injecting therein a fluid, thus transmitting a pressure to tissues of the tumoral mass; and
delivering high frequency electromagnetic energy to the tumoral mass until coagulative necrosis of the tissues of the tumoral mass;
wherein a pressure transmitted by the expandable balloon to the tissues of the tumoral mass is higher than atmospheric pressure,
the method further including the steps of
measuring and controlling said transmitted pressure.

36. The method of claim 35, wherein the measuring is performed through transducers and the controlling is performed based on a pressure value detected by said transducers.
